(19) United States
(12) Patent Application Publication
(10) Pub. No.: US 2012/0172963 A1
(43) Pub. Date: Jul. 5, 2012

(54) DELIVERY DEVICE

(76) Inventors: Michael Ryan, Limerick (IE); John Neilan, Galway (IE); Donagh O'Sullivan, Limerick (IE); Fionan Keady, Galway (IE)

(21) Appl. No.: 13/346,220

(22) Filed: Jan. 9, 2012

Related U.S. Application Data
(63) Continuation-in-part of application No. 11/879,176, filed on Jul. 16, 2007.
Application No. 11/879,176 is a continuation-in-part of application No. 13/015,764, filed on Jan. 28, 2011.
(60) Provisional application No. 61/434,245, filed on Jan. 19, 2011.
(60) Provisional application No. 61/434,267, filed on Jan. 19, 2011.

(51) Int. Cl. A61F 2/82

(52) U.S. Cl. 623/1.11

(57) ABSTRACT

A delivery device for deploying an expandable prosthesis and method of use thereof are described. The delivery device includes an outer sheath that is capable of retracting in a proximal direction and resheathing over the prosthesis in a distal direction. The device includes a drive pulley that can engage gears to retract or resheath the outer catheter in relation to the prosthesis. In some embodiments, the delivery device may include a reinforced outer sheath disposed over an inner elongate member, the reinforced outer sheath comprising a proximal section reinforced with a braid, a distal section reinforced with a coil and an overlapping section extending between the proximal section and the distal section. Additionally or alternatively, the delivery device may include a stabilizing element for releasably holding the stent to the inner catheter.

### CROSS-REFERENCE TO RELATED APPLICATIONS

This application claims priority to both U.S. Provisional Patent Application Nos. 61/434,245, and 61/434,267, both filed Jan. 19, 2011, and is a continuation-in-part of both pending U.S. patent application Ser. Nos. 11/879,176, filed Jul. 16, 2007, and 13/015,764, filed Jan. 28, 2011, each of which is incorporated herein by reference in its entirety.

### TECHNICAL FIELD

This invention relates to a medical device and, in particular to a delivery device for a self-expanding prosthesis and a method of delivering and deploying the prosthesis into a body lumen.

### BACKGROUND

A self-expanding prosthesis (e.g., a stent) typically is introduced into the body using a delivery device that includes a push-pull mechanism. The typical push-pull delivery device includes an outer catheter coaxially disposed and slidable over an inner catheter. The prosthesis is disposed at the distal end of the device in between the inner catheter and the outer catheter. The inner and the outer catheter move coaxially with respect to each other such that the prosthesis may be deployed by proximally pulling back the outer catheter relative to the inner catheter until the prosthesis is exposed.

There are numerous drawbacks to the above push-pull delivery device. For example, utilizing a conventional push-pull delivery device may cause the physician inadvertently to use excessive force and pull back the outer catheter too far, thereby prematurely deploying the prosthesis in an incorrect position within a body lumen. Subsequent repositioning of the prosthesis may be difficult, if not impossible, because the prosthesis will already have radially self-expanded into the body lumen. Additionally, retraction of the outer sheath is not achieved with controlled movement because the physician is manually retracting the outer catheter. Manual retraction of the outer catheter may lead to inadvertent jerking back of the outer catheter. Furthermore, two hands are typically needed to deploy the prosthesis with a push-pull mechanism. One hand may be required to hold the inner catheter while the other hand pulls the outer catheter and slides it back over the inner catheter. This requirement for using both hands prevents the physician from performing another task during the procedure, and may necessitate the presence of other personnel for procedures increasing logistical complexity and cost.

Accordingly, in view of the drawbacks of current technology, there is a desire for a delivery system that can increase the control, accuracy and ease of placement during deployment of a prosthesis. Although the inventions described below may be useful for increasing the control, accuracy and ease of placement during deployment of the prosthesis, the claimed inventions may also solve other problems.

### SUMMARY

Accordingly, a delivery device is provided comprising an outer catheter that is capable of retracting in a proximal direction and resheathing over the prosthesis in a distal direction.

The invention may include any of the following aspects in various combinations and may also include any other aspect described below in the written description or in the attached drawings.

In one aspect, a stent delivery device may include a threaded drive member that simultaneously moves inner and outer shafts in a manner configured to deploy and/or resheath a stent.

### BRIEF DESCRIPTION OF THE DRAWINGS

Embodiments will now be described by way of example with reference to the accompanying drawings, in which:

FIG. 1 is a perspective view of a delivery device;

FIG. 2 is a perspective view of a first gear set of the delivery device;

FIG. 3 is a perspective view of a second gear set of the delivery device;

FIG. 4 is a perspective view of the delivery device showing the outer catheter connected to a belt;

FIG. 5 shows a shuttle cap being threadedly attached to the shuttle to secure the outer catheter to the shuttle;

FIG. 6 shows the attachment of the belt to the shuttle and outer catheter;

FIG. 7 shows protrusions on one of the faces of the pulley gear that is configured to slot into corresponding slotted ribs located on the center drive pulley;

FIG. 8A shows the trigger, drive gears and pulley gears;

FIG. 8B shows an enlarged view of one embodiment of a directional switch;

FIGS. 9-10 shows ribbed slots on the center drive pulley (FIG. 10) that are configured to receive the pulley gears (FIG. 9);

FIG. 11-11A show the trigger alone, and with drive gears, respectively;

FIG. 12 shows the entire delivery device preloaded with an esophageal stent at the distal tip of the delivery section;

FIGS. 13-16 show a method of use of the delivery device;

FIG. 17 shows a main drive gear rotationally fixed to the drive axle;

FIGS. 18A-18C show an internal mechanism embodiment of a stent deployment/resheathing device; and

FIG. 19 shows another internal mechanism embodiment of a stent deployment/resheathing device.

### DETAILED DESCRIPTION OF THE PREFERRED EMBODIMENTS

The embodiments are described with reference to the drawings in which like elements generally are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

Throughout the specification, the terms “distal” and “distally” shall denote a position, direction, or orientation that is generally away from the physician. Accordingly, the terms “proximal” and “proximally” shall denote a position, direction, or orientation that is generally towards the physician.

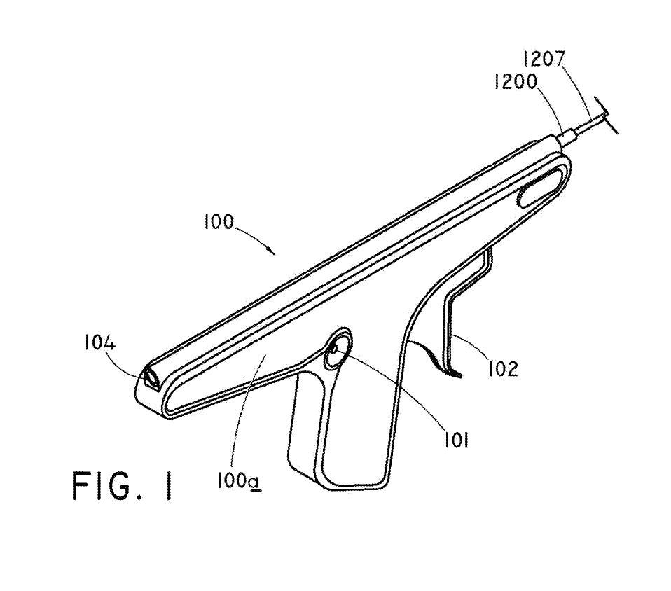

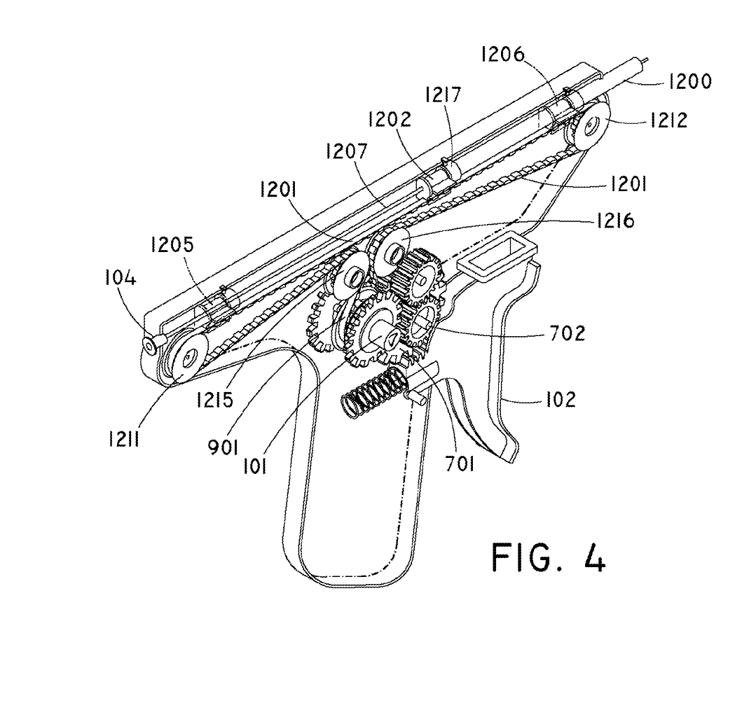

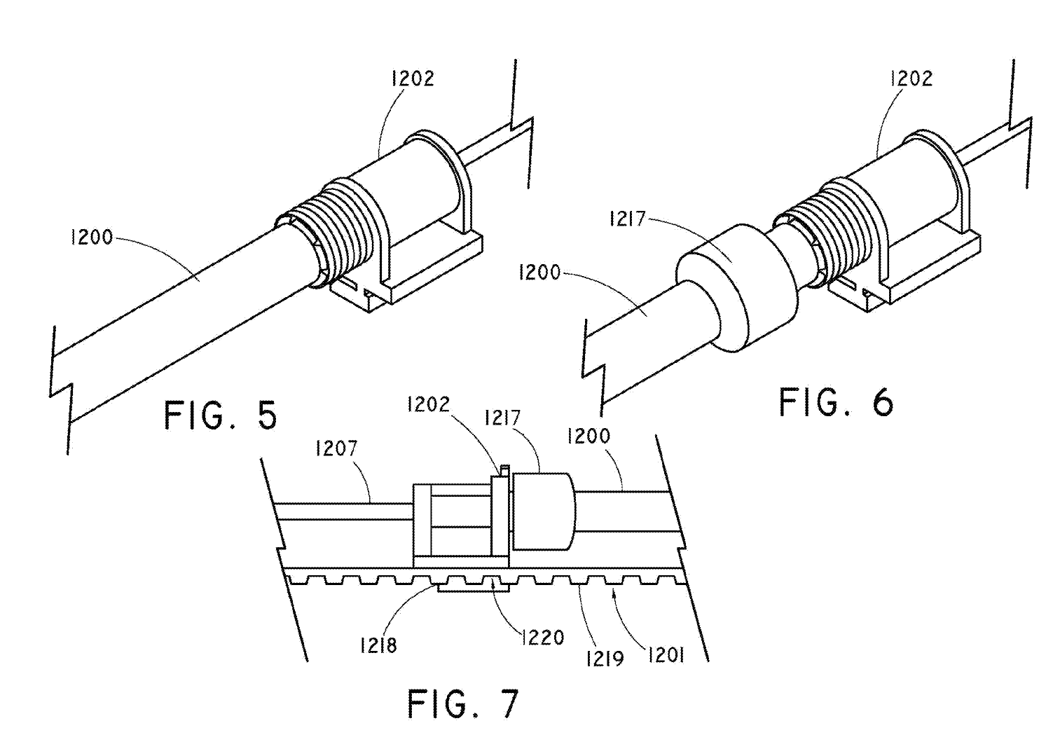

Referring now to the drawings in FIGS. 1-19, a delivery device for deploying a self-expanding prosthesis is shown. As will be discussed, the delivery device has the ability to resheath and reposition the prosthesis, thereby substantially increasing the control and accuracy of the deployment process as compared with conventional delivery devices. Mechanisms for attaching, releasing, deploying, and resheathing of stent devices are also disclosed in U.S. Pat. App. No. 61/299,605, filed Jan. 29, 2010, which is incorporated herein by reference in its entirety.

FIG. 1 shows an exemplary delivery device 100, the workings of which are here explained with reference to FIGS. 1-12. The inner catheter 1207 and outer catheter 1200 are shown exiting the distal end of the device 100. In the embodiments of FIGS. 1-17, the inner catheter 1207 remains fixated to the delivery device 100 at the rear hub 104. As described herein, several different embodiments of internal actuation mechanisms may be operable within a “gun-shaped” housing that is similar to the housing 100a in FIG. 1; however, each of the embodiments herein described may occupy housing of differing external appearance. The outer catheter 1200 may be affixed to a movable belt 1201 (FIG. 4). Actuation of a spring-loaded trigger 102 pulls the outer catheter 1200 in the proximal direction relative to the inner catheter 1207 to expose the self-expanding prosthesis. A directional switch 101 may be engaged to reverse the direction of the outer catheter 1200 prior to actuating the trigger 102. An internal gear-pulley mechanism enables the bidirectional movement of the outer catheter 1200.

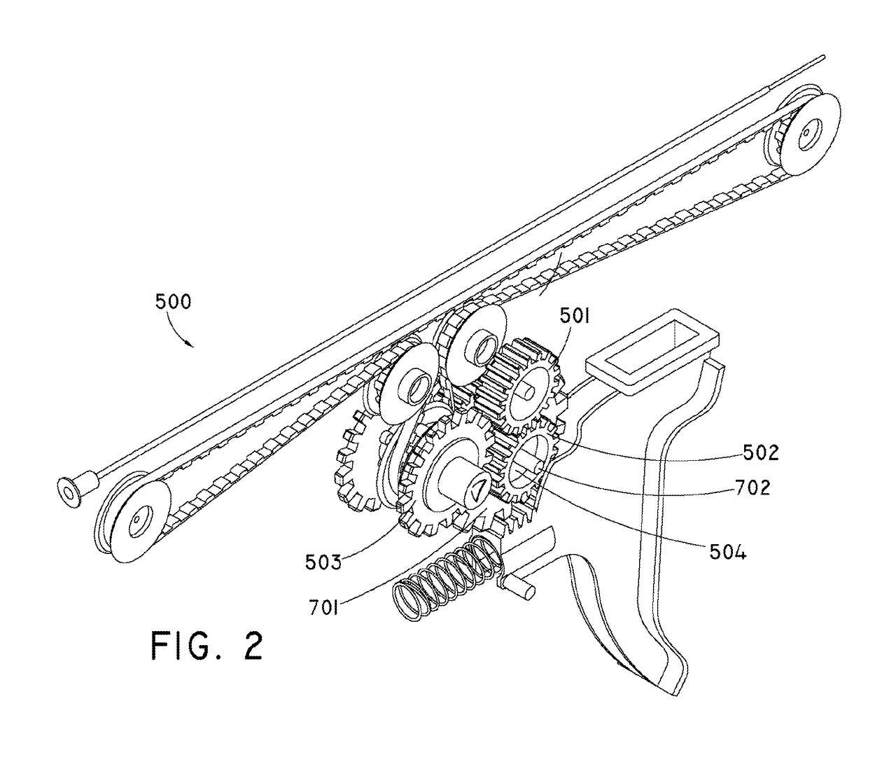

A first gear set is configured to resheath the outer catheter 1200 over the inner catheter (and—potentially—a prosthesis overlying the inner catheter) by moving the outer catheter 1200 in a distal direction relative to the inner catheter 1207, and a second gear set is configured to retract the outer catheter 1200 (i.e., move the outer catheter 1200 in a proximal direction relative to the inner catheter 1207). FIG. 2 shows the first gear set 500. The first gear set 500 includes a first drive gear 502, a first idle gear 501, and a first pulley gear 503. The first drive gear 502 is mechanically engaged with the first idle gear 501. The first idle gear 501 is mechanically engaged with the first pulley gear 503. The first drive gear 502 has a one-directional roller clutch bearing 504. Specifically, the roller clutch bearing 504 is press fit within the inner surface of the first drive gear 502 and allows for rotation of the first drive gear 502 in only one direction, which will be explained in greater detail below.

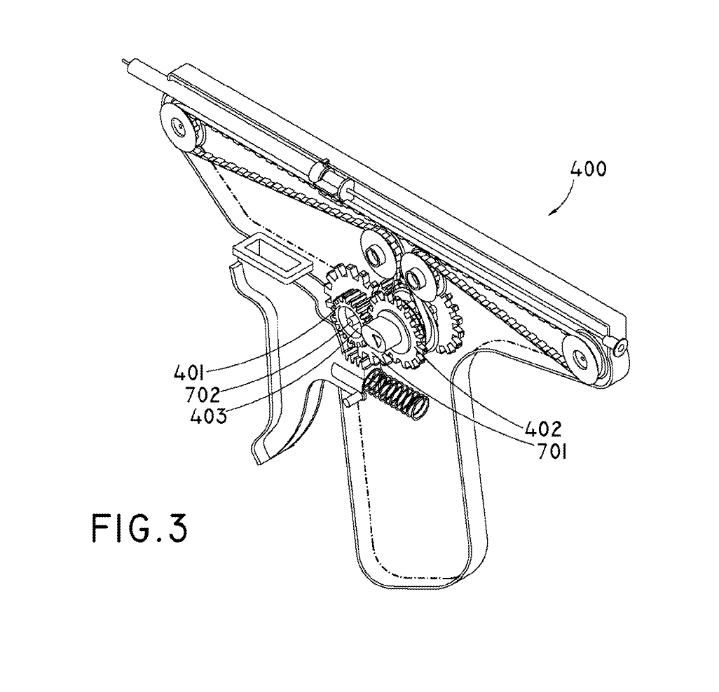

FIG. 3 shows the second gear set 400. The second gear set 400 includes a second drive gear 401 and a second pulley gear 402. The second drive gear 401 is mechanically coupled to the second pulley gear 402. Similar to the first drive gear 502, the second drive gear 401 also includes a roller clutch bearing 403 that allows for rotation of the gear 401 in only one direction, which will be explained in greater detail below.

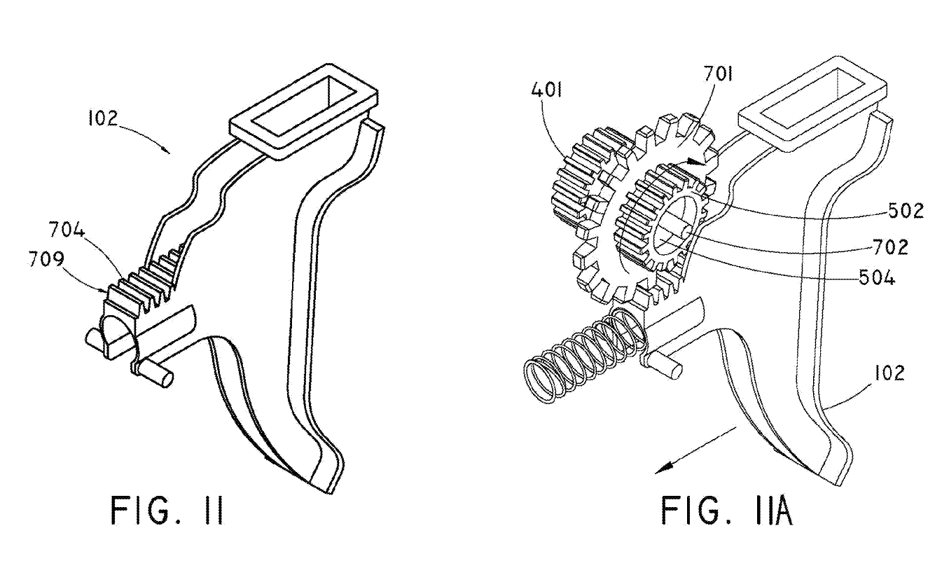

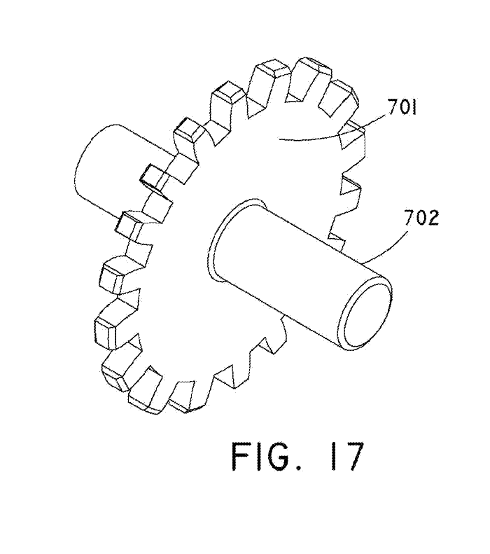

A drive axle 702 extends through the clutch bearing 403 of the second drive gear 401 (FIG. 3) and through the clutch bearing 504 of the first drive gear 502 (FIG. 2). A main drive gear 701 is rotationally fixed to the drive axle 702, which is more clearly shown in FIG. 17. The main drive gear 701 is also engaged with a trigger 102 (FIG. 11). The trigger 102 includes a rack 709 having complimentary teeth 704 (FIG. 10) that engage with the main drive gear 701.

Proximal and distal movement of the outer catheter 1200 may be effected by the outer catheter 1200 being connected by a belt 1201 to the gearing mechanism of FIGS. 2-3, as shown in FIG. 4. The outer catheter 1200 is affixed to a shuttle 1202 and the shuttle 1202 is connected to a belt 1201. FIG. 6 shows how the outer catheter 1200 may be affixed to the shuttle 1202 by being flared and pushed up against the shuttle 1202. After abutting the flared end of the outer catheter 1200 against the shuttle 1202, FIGS. 6-7 show that a shuttle cap 1217 may be coupled to the shuttle 1202. Specifically, the cap 1217 may be threadedly attached onto the threads of the shuttle 1202 to secure the outer catheter 1200 to the shuttle 1202. The inner catheter 1207 may be secured to the rear hub 104 in a similar manner. Other types of attachments of the outer catheter 1200 to the belt 1201 are contemplated.

The attachment of the belt 1201 to the shuttle 1202 and outer catheter 1200 may be seen in FIG. 7. FIG. 7 shows that the shuttle 1202 contains an opening 1218 through which belt 1201 may extend. The shuttle 1202 contains corresponding grooves 1220 that engage with protrusions 1219 of the belt 1201 to establish a secure belt-shuttle connection. Movement of the belt 1201 causes the shuttle 1202 and outer catheter 1200 attached thereto to laterally move along the belt 1201 in the proximal direction or distal direction.

Referring to FIG. 4, activation of the first gear set 500 or the second gear set 400 rotates a center drive pulley 901 and the belt 1201 to cause the shuttle 1202 with the outer catheter 1200 attached thereto to move with the belt 1201. FIG. 4 illustrates possible positions that the outer catheter 1200 may have, although the trigger 102 is not shown as moving. The most proximal position of the shuttle 1202 and belt 1201 is indicated at position 1205. The most distal position of the shuttle 1202 and belt 1201 is indicated at position 1206. For purposes of clarity, the shuttle cap 1217 is not shown at positions 1205 and 1206. As the outer catheter 1200 moves along the belt 1201, the inner catheter 1207 of this embodiment remains stationary because the inner catheter 1207 is fixated at the proximal end of the device 100 at the rear hub 104.

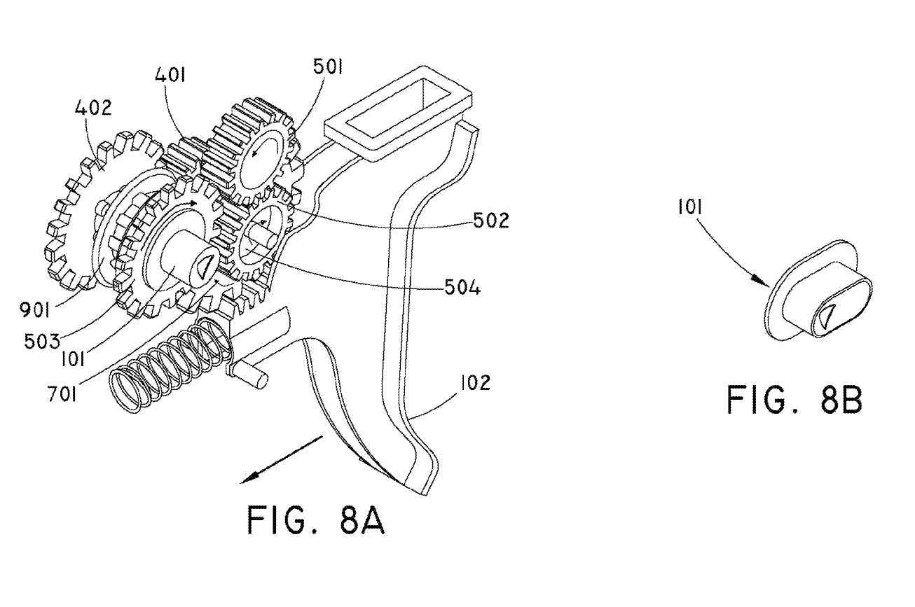

With reference to FIGS. 7-10, it should be appreciated that desired belt 1201 movement may be achieved by engaging a center drive pulley 901 with the first pulley gear 503 or the second pulley gear 402. The first pulley gear 503 and the second pulley gear 402 are slidable along a shaft to engage and disengage with the drive pulley 901. The engagement and disengagement may occur by the ribs or protrusions 1000 of the pulley gears 503, 402 slidably engaging with the ribbed slots 902 of the center drive pulley 901. Directional switch 101 allows the first pulley gear 503 or the second pulley gear 402 to engage with the center drive pulley 901. FIG. 8B illustrates an exemplary directional switch 101. Referring to FIG. 8A, the first pulley gear 503, second pulley gear 402, and directional switch 101 extend along a shaft (not shown). Pushing the directional switch 101 against the first pulley gear 503 causes the first pulley gear 503 to engage with the center drive pulley 901 and the second pulley gear 402 to disengage with the center drive pulley 901 along the shaft. At any given time, the center drive pulley 901 may be engaged to either the first pulley gear 503 or the second pulley gear 402.

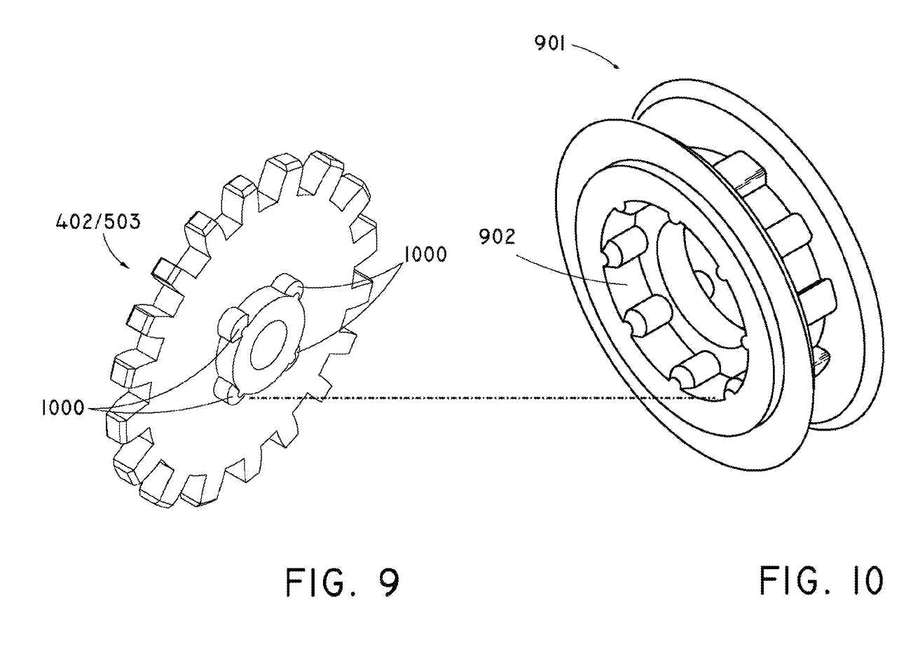

The engagement of the first or second pulley gears 503, 402 with the center drive pulley 901 can be understood by referring to FIGS. 8A and 9 with reference also to FIG. 3. The first and second pulley gears 503 and 402 may appear as shown in FIG. 8A. FIG. 10 shows that the center drive pulley 901 contains ribbed slots 902 that correspond to protrusions 1000 (FIG. 9) of the first and second pulley gears 503, 402. The multiple side protrusions 1000 of the first and second pulley gears 503, 402 (FIGS. 8A, 9) slide into the ribbed slots 902 located on the side of the center drive pulley 901 (FIG. 10) to lockably engage with each other. The engagement may be such that when the locked first pulley gear 503 or locked second pulley gear 402 rotates, the center drive pulley 901 will rotate in the same direction, thereby transferring the motion of the pulley gears 503, 402 to the drive pulley 901 and belt 1201.

The first and second pulley gears 503 and 402 may include a greater or lesser number of ribbed slots 902 than that shown in FIG. 8 to facilitate engagement of the pulley gears 503 and 402 with the center drive pulley 901. Alternatively, or in addition, the shape of the ribbed slots 902 of the center drive pulley 901 may be modified to enhance its engagement with the gears 503 and 402.

The belt 1201 is shown in FIG. 4 as wrapped around three pulleys 1211, 1212 and 901. Pulleys 1211 and 1212 may help transfer gear movement into belt movement. Center drive pulley 901 engages with one of the first gear set 500 and the second gear set 400 to cause rotational movement of the belt 1201. Although a three pulley system is shown, more than three pulleys or less than three pulleys are contemplated.

Idlers 1215 and 1216 (FIG. 4) may help to provide wrapping a sufficient amount of the belt 1201 around the center drive pulley 901 for the purpose of preventing belt 1201 slippage from the center drive pulley 901. Referring to FIG. 4, the belt 1201 wraps around idler 1215 and then proceeds down and around the center drive pulley 901. The belt 1201 then proceeds up and around the top of idler 1216. FIG. 4 shows that the idlers 1215, 1216 help the belt 1201 to wrap around more than 180° of the center drive pulley 901.

The gear mechanism for resheathing (i.e., moving the outer catheter 1200 from a proximal direction to a distal direction) will now be explained. Reference to the rotational movement of the various gears and pulleys will be made in accordance with perspective views facing the first gear set 500 (FIGS. 4, 8A, 9, 10). The directional switch 101 is pushed such that the first pulley gear 503 is engaged with the center drive pulley 901 and the second pulley gear 402 is disengaged from the center drive pulley 901 (FIG. 8A). Pulling the trigger 102 in the proximal direction, as indicated by the arrow in FIG. 8A, rotates the main drive gear 701, which is engaged with the rack 709 (FIGS. 11-11A) of the trigger 102 in a clockwise direction (see the motion arrow in FIG. 11A around first drive gear 502). Because the main drive gear 701 is directly connected to the drive axle 702, the drive axle 702 also rotates in a clockwise direction. As the drive axle 702 rotates in a clockwise direction, the first drive gear 502 and the second drive gear 401 also rotate in the same direction. The first drive gear 502 is engaged to the first idle gear 501 and therefore clockwise rotation of the first drive gear 502 causes the first idle gear 501 to rotate counterclockwise (see FIG. 8A). The first idle gear 501 is engaged to a first pulley gear 503. Accordingly, counterclockwise rotation of the first idle gear 501 causes the first pulley gear 503 to rotate clockwise (FIG. 8A). Because the directional switch 101 has been pushed to engage the first pulley 503 with the center drive pulley 901 (FIG. 8A), the center drive pulley 901 also rotates in the clockwise direction. With the belt 1201 winding around a center drive pulley 901, two idlers 1215 and 1216 gather the belt 1201 around the center drive pulley 901, as shown in FIG. 4. The idlers 1215 and 1216 optimize the connection between the belt 1201 and the center drive pulley 901 to minimize slippage of the belt 1201 around the center drive pulley 901. Clockwise rotation of the center drive pulley 901 also causes the belt 1201 to move generally clockwise (in the perspective shown in FIG. 4). The generally clockwise motion of the belt 1201 will cause the shuttle 1202 and outer catheter 1200 attached thereto to resheath (that is, to move distally over the inner catheter).

When the trigger 102 has been deactivated so that the trigger 102 moves distally and returns to its original position, the drive axle 702 and main drive gear 701 rotate counterclockwise and return to their original position as a result of action by the trigger-biasing spring 771 (which is configured to keep the trigger 102 oriented in a default position when not being depressed/actuated. The drive axle 702 is permitted to rotate counterclockwise within the one-directional roller clutch bearings 403, 504. However, roller clutch bearings 403, 504 prevent the left and right drive gears 401, 502 from rotating counterclockwise upon the trigger 102 being deactivated. Thus, the first and second drive gears 502 and 401 will remain in the position from which they have rotated clockwise after activation of the trigger 102. The effect of having the first drive gear and the second drive gears 502 and 401 rotate clockwise but not counterclockwise is that the outer catheter 1200 may continue to be incrementally moved in a proximal (i.e., retractable direction) or distal direction (i.e., resheathing direction). Accordingly, this unidirectional movement of the first and second drive gears 502 and 401 is converted into movement of the belt 1201.

The gear mechanism for retracting the outer catheter 1200 will now be explained. Reference to the rotational movement of the various gears and pulleys will be made in accordance with perspective views facing the second gear set 400 (see, e.g., FIG. 3). The directional switch 101 is pushed such that the second pulley gear 402 is engaged with the center drive pulley 901 and the first pulley gear 503 is disengaged from the center drive pulley 901. Referring to FIG. 3, pulling the trigger 102 in a proximal direction causes the main drive gear 701, engaged with the rack 709 of the trigger 102, to rotate in a counterclockwise direction. Because the main drive gear 701 is directly connected to the drive axle 702, the drive axle 702 also rotates in a counterclockwise direction. As the drive axle 702 rotates in a counterclockwise direction, the first drive gear 502 and the second drive gear 401 rotate in the same direction. Because the second drive gear 401 is engaged to the second pulley gear 402, counterclockwise rotation of the second drive gear 402 causes the second pulley gear 402 to rotate clockwise (see FIG. 3). The engagement of the second pulley gear 402 with the center drive pulley 901 causes the center drive pulley 901 to also rotate in a clockwise direction (see FIG. 3).

Referring to FIGS. 3-4, the rotation of the second pulley gear 402 with the center drive pulley 901, which was seen as clockwise from the perspective in FIG. 2, becomes viewed as counterclockwise from the perspective in FIG. 3. The counterclockwise rotation of the center drive pulley 901 also causes the belt 1201 to rotate counterclockwise. The counterclockwise rotation of the belt 1201 causes the shuttle 1202 and outer catheter 1200 attached thereto to retract or move distally to proximally, thereby exposing the self-expanding prosthesis. A step may be formed where the smaller and larger diameter portions of the inner catheter 1207 meet, which prevents the prosthesis from being pulled back proximally with the outer sheath 1200.

The unidirectional movement of the first and second drive gears 502 and 401 is converted into proximal movement of the belt 1201 and outer catheter 1200 attached thereto. Specifically, when the trigger 102 has been deactivated so that the trigger 102 moves distally and returns to its original position, the drive axle 702 and main drive gear 701 rotate clockwise with respect to FIG. 3 and return to their original position. The drive axle 702 is permitted to rotate clockwise within the one-directional roller clutch bearings 403, 504. However, roller clutch bearings 403, 504 prevent the left and right drive gears 401, 502 from rotating upon the trigger 102 being deactivated. The effect of having the first drive gear and the second drive gears 502 and 401 rotate counterclockwise but not clockwise (as shown in FIG. 3) is that the outer catheter 1200 may continue to be incrementally moved in a proximal direction (i.e., retractable direction).

In order to prevent the self-expanding prostheses from moving as the outer catheter 1200 moves during resheathing, a stabilizing element may be affixed to the prosthesis. The stabilizing element will maintain the prosthesis in a substantially stationary position during the resheathing of the outer catheter 1200 over the prosthesis, as will now be explained. Various types of stabilizing elements may be used within the scope of the present invention, such as—for example—those disclosed in U.S. Pat. App. Publ. No. 2010/0168834, which is incorporated herein by reference in its entirety.

Delivery of through-the-scope (TTS) self-expandable stents configured for deployment through an endoscope working channel within the gastrointestinal tract necessitates that the outer sheath and inner catheter be sufficiently small in size to fit through an accessory channel of an endoscope. Additionally, because tumors within the gastrointestinal tract often are situated in difficult-to-access regions (e.g., ascending colon or duodenum), the outer sheath and inner catheter should be sufficiently flexible, yet kink resistant and pushable, to navigate to such difficult-to-access regions. Notwithstanding these desirable attributes of the outer sheath and inner catheter, the extent to which the lateral profile of the outer sheath may be decreased may be limited by the radial force the TTS self-expandable stent is required to exert at the target stricture.

Outer sheaths that are too thin may not have sufficient mechanical strength to deploy a TTS stent because the TTS stent should exert a radial force sufficient to maintain patency at the stricture and remain anchored therewithin so as to be resistant to any tendency to migrate away from the stricture due to peristalsis effects. Therefore, generation of sufficient radial force at a target stricture requires deploying a TTS stent with a strong radial force. Accordingly, thin sheaths may experience higher stress levels during deployment (i.e., the forces required at the handle of the device 100 to proximally pull the outer sheath 1200 relative to inner catheter 1207 to fully expose the stent 2804) and during resheathing (i.e., the forces required at the handle of the device 100 for distally pushing the outer sheath 1200 relative to the inner catheter 1207 to fully resheath the inner catheter 1207) as compared to larger sheaths. The higher forces required for resheathing or deployment of outer sheath can be burdensome. Although larger sized outer sheaths may be used to decrease such forces, the outer diameter of the outer sheath is limited by the size of the accessory channel on the endoscope, which concordantly reduces the inner diameter of the outer sheath and thereby limits the amount of radial force of a TTS stent that can be loaded and deployed. Accordingly, an outer sheath preferably is selected so as to achieve a balance between the above described limitations. For example, various reinforced sheath designs may be used, such as embodiments described in U.S. Pat. App. Publ. No. 2010/0168834.

Having described the structure and operation of the device 100 (i.e., the internal gear mechanism to retract/resheath the outer catheter 1200), a method of use of the device 100 is now described with reference to FIGS. 3, 4, and 12-16. The device 100 may be used to deploy various prostheses. As an example, a method of deploying an esophageal stent 301 will now be described. The esophageal stent 301 is loaded in between the inner catheter 1207 and the outer catheter 1200 along the distal end 1700 of the device 100, as shown in FIG. 12. Part of the loading process of the stent 301 may include affixing a retaining wire from one of the crowns at the proximal end of the stent 301 to the rear hub 104 located at the proximal end of the device 100.

Delivery and deployment are described here with reference to the esophageal stent 301; however, one skilled in the art will understand that the delivery and deployment methods are also applicable to other prosthesis and deployment device embodiments than are described herein. The delivery and deployment process may begin after having loaded the esophageal stent 301 and affixed a retaining wire or other retaining means to the esophageal stent 301. The delivery device 100 includes a flexible stent-delivery section 1702 and an external manipulation section 1703. The delivery section 1702 is directed through the body lumen during the procedure and delivers the prosthesis to a desired deployment site within the esophagus. The external manipulation section (actuation handle) 1703 will remain outside of the body during the procedure. The external manipulation section 1703 includes trigger 102 and may be manipulated by the physician with a single hand (see FIG. 13) to position and release the stent 301 into the body lumen.

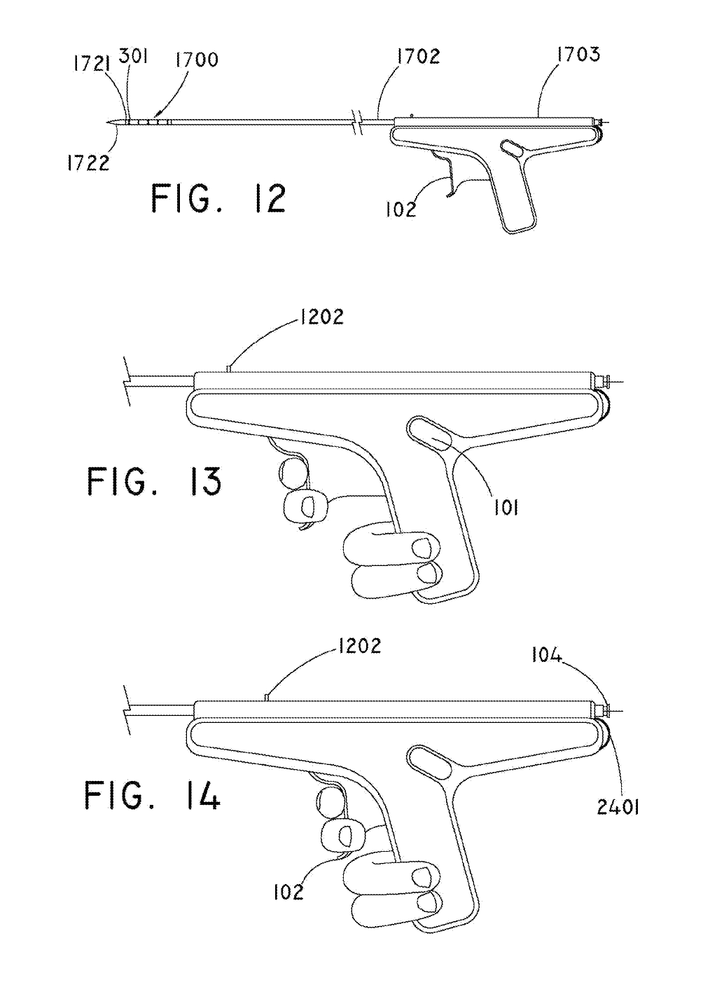

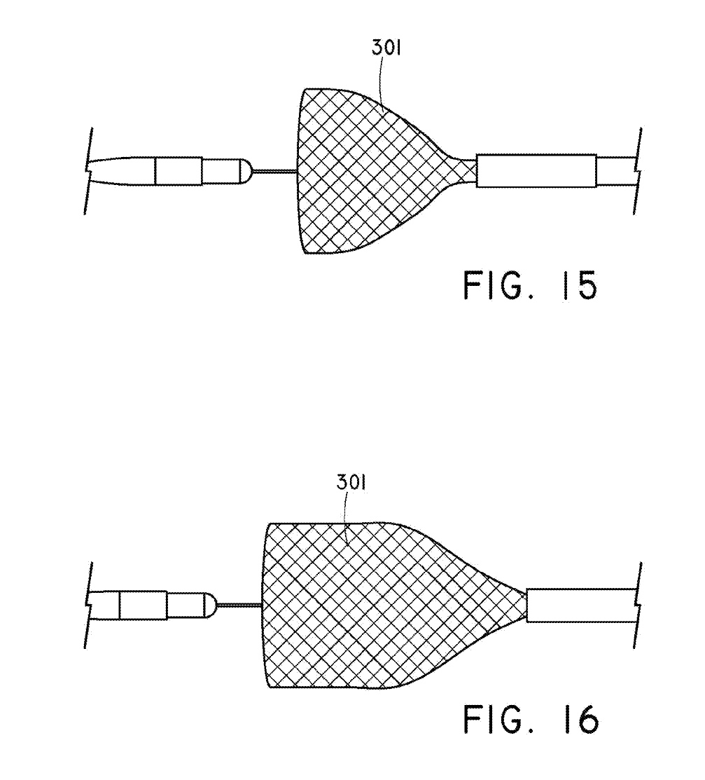

After having delivered the delivery section 1702 of the delivery device 100 to the target site within the esophagus, the deployment of the stent 301 may begin. The trigger portion 102 of the device 100 will remain outside of the patient to enable deployment of the esophageal stent 301. The physician will depress the directional switch 101 across the long axis of the actuation handle 1703 to actuate the second gear set 400 (see FIG. 3) to enable proximal retraction of the outer catheter 1200 relative to the inner catheter 1207. FIG. 13 shows that the shuttle 1202 is positioned near the distal end of the external manipulation section 1703. Having pressed the directional switch 101 to actuate the second gear set 400 with the center drive pulley 901, the physician may grasp the trigger 102 of the device 100 with a single hand, as shown in FIG. 13, to actuate the trigger 102 for the first time. The other hand may be free to perform other tasks. FIG. 14 shows that the trigger 102 has been completely pulled backed in the proximal direction. In particular, the tip of the shuttle 1202 has proximally moved after one actuation of the trigger 102. With the second pulley gear 402 still mechanically coupled to the center drive pulley 901, trigger 102 is actuated multiple times to retract the outer catheter 1200 in the proximal direction relative to the inner catheter 1207 until a portion of the esophageal stent 301 has become exposed and partially radially expanded, as shown in FIG. 15. Further actuations of the trigger 102 cause the outer sheath 1200 to proximally move back even further, thereby exposing an increasing portion of the self-expanding stent 301, as shown in FIG. 16.

At this juncture, notwithstanding partial radial expansion of the stent 301, the device 100 may be activated to resheath the outer catheter 1200 over the stent 301 to allow repositioning of the stent 301 within the esophagus. The physician may need to resheath and reposition the stent 301 as a result of having placed the stent 301 in the incorrect position. The directional switch 101 may be pressed to disengage the center drive pulley from the second pulley gear and to engage the center drive pulley with the first pulley gear (FIG. 8A). Having activated the first gear set 500 with the center drive pulley 901, actuation of the trigger 102 one or more times will move the outer sheath 1200 distally and thereby resheath over the stent until the stent 301 is fully constrained back within the outer sheath 1200. With the stent 301 fully recaptured within the outer catheter 1200, the external manipulation section 1703 may be maneuvered to reposition the delivery section 1702 within the body lumen. After repositioning the delivery section 1702, the directional switch 101 may be reconfigured to reactivate the second gear set 400 with the center drive pulley 901 such that proximal retraction of the outer sheath 1200 occurs, thereby exposing the stent 301. A retaining wire or other retaining means retains the stent 301 and prevents it from moving distally during resheathing.

Referring to FIG. 12, during deployment, the distal end 1700 of the outer catheter 1200 may include a transparent or translucent material (or a light-transmitting material) to enable the physician to visually observe the stent 301 and how it is positioned in relation to the esophageal stricture. FIGS. 13-14 show that the top-most portion of the shuttle 1202 protrudes through the housing of the device 100. The top-most portion of the shuttle 1202, moves proximally as the outer catheter 1200 is retracted proximally and may be used as a visual indicator to determine when resheathing capabilities have been lost. The distance that the top-most portion of the shuttle 1202 moves proximally corresponds to the distance that the outer catheter 1200 has been retracted. The top-most portion of the shuttle 1202 can move proximally a predetermined threshold distance beyond which the physician will realize that the outer catheter 1200 cannot be proximally retracted any further without losing the ability to resheath and recapture the stent 301 within the outer catheter 1200. Alternatively, the point at which the top-most portion of the shuttle 1202 aligns with a predetermined visual marker on the outer housing of the device 100 can also indicate the loss of the ability to resheath.

One or more radiopaque markers 1721 may be used under fluoroscopy to determine the distance the outer catheter 1200 has proximally retracted (FIG. 12). The radiopaque marker 1721 may be placed on the outer catheter 1200 between the distal tip 1722 and the distal end 1700 of the clear portion of the outer catheter 1200, as shown in FIG. 12. The one or more markers 1721 may be utilized to determine when the resheathing capabilities have been lost. For example, as the outer catheter 1200 is proximally retracted, the radiopaque marker 1721 may move along with it. The marker on the inner catheter 1207 (FIG. 1) may be positioned such that if the marker 1721 on the outer catheter 1200 aligns with the marker on the inner catheter 1207, the physician will realize that the stent 301 cannot be exposed any further without losing the ability to resheath and recapture the stent 301 within the outer catheter 1200.

As can be seen, the device 100 is capable of incrementally deploying the stent 301. In the above examples described, one full actuation of the trigger 102 may proximally move the belt 1201 and hence the outer sheath 1200 from about 5 mm to about 10 mm. Such incremental deployment may facilitate greater accuracy in positioning of the stent 301 at the target region. On the contrary, a conventional push-pull delivery device has less control as compared to the delivery device 100 because the conventional push-pull delivery device cannot withdraw the outer sheath in such small, precise increments. Conventional push-pull delivery devices require the user to maintain one portion of the handle in a fixed position and manually either pull in a proximal direction relative to the fixed portion of the handle or push in a distal direction relative to the fixed portion of the handle to resheath the stent. The speed and control of the pulling and pushing of such conventional push-pull delivery devices is wholly dependent on the user, thereby preventing deployment in the small, precise increments which device 100 can perform. Additionally, stents with low or high deployment forces may contribute to the lack of control of push-pull delivery devices. The lack of control may result in sudden proximal movement of the outer sheath of about 50 mm or more, resulting in inaccurate placement of the deployed stent.

Another advantage of the device 100 as has been described is the ability to resheath the outer catheter 1200 over the stent 301. The resheathing feature gives the physician the ability to make real-time adjustments during the deployment procedure such that the stent may be repositioned. In the examples described, the stent 301 may be able to be resheathed even after about 10% of the stent 301 has been deployed or as much as about 95% of the stent 301 has been deployed. Yet other advantages include the ability to use a single hand to deploy the stent 301. The other hand may be free to perform other tasks, such as holding an endoscope when deploying a self-expandable stent therethrough.

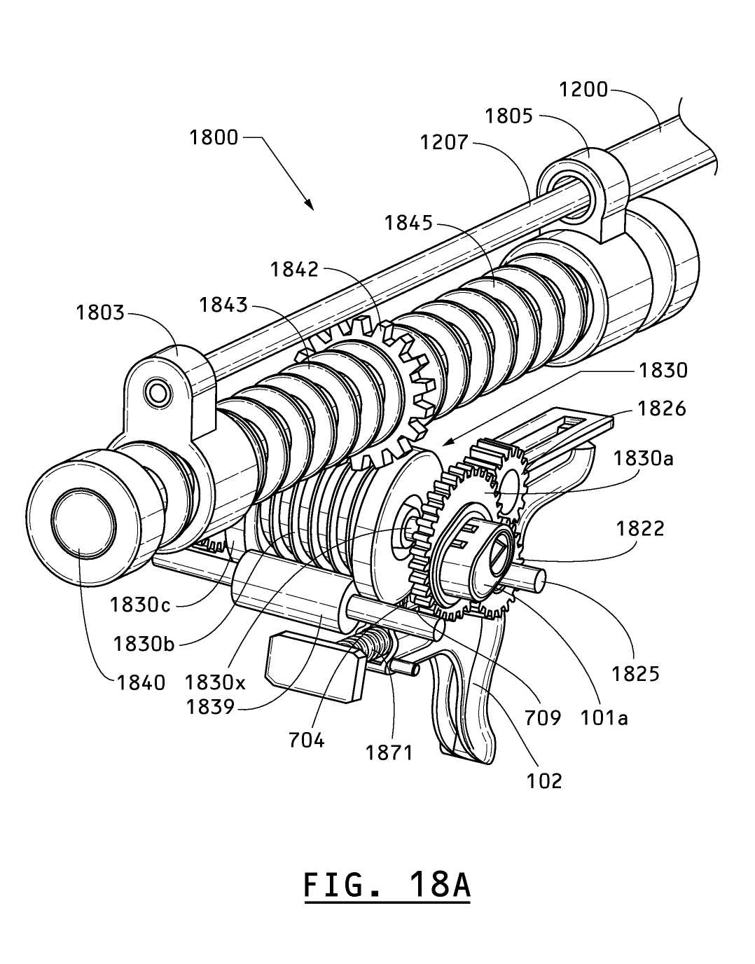

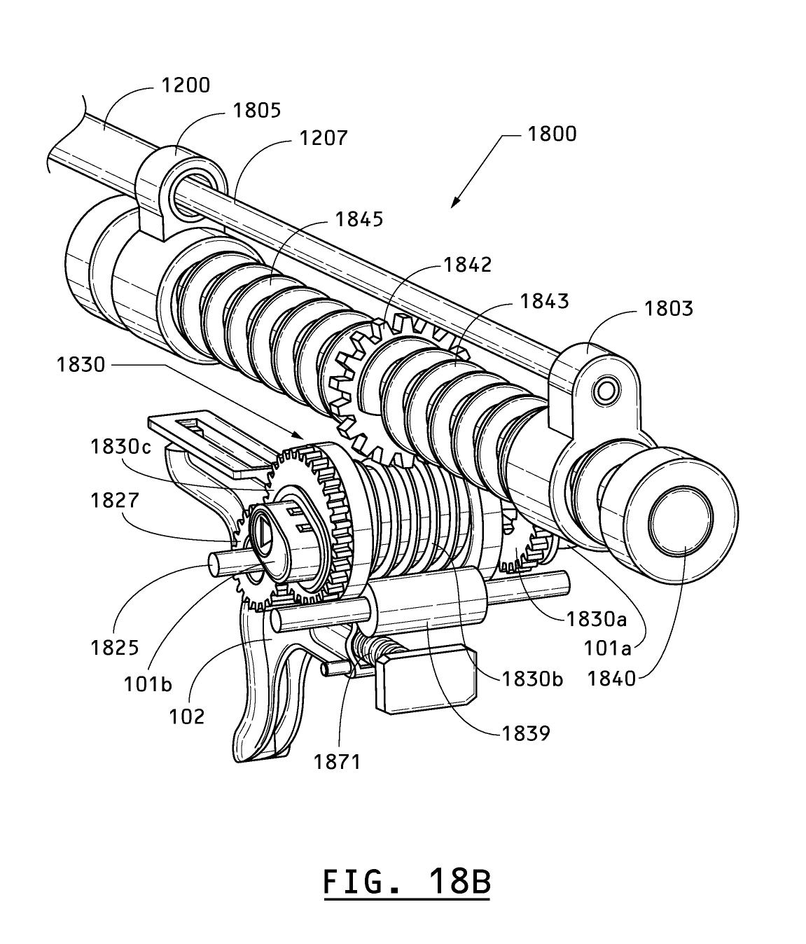

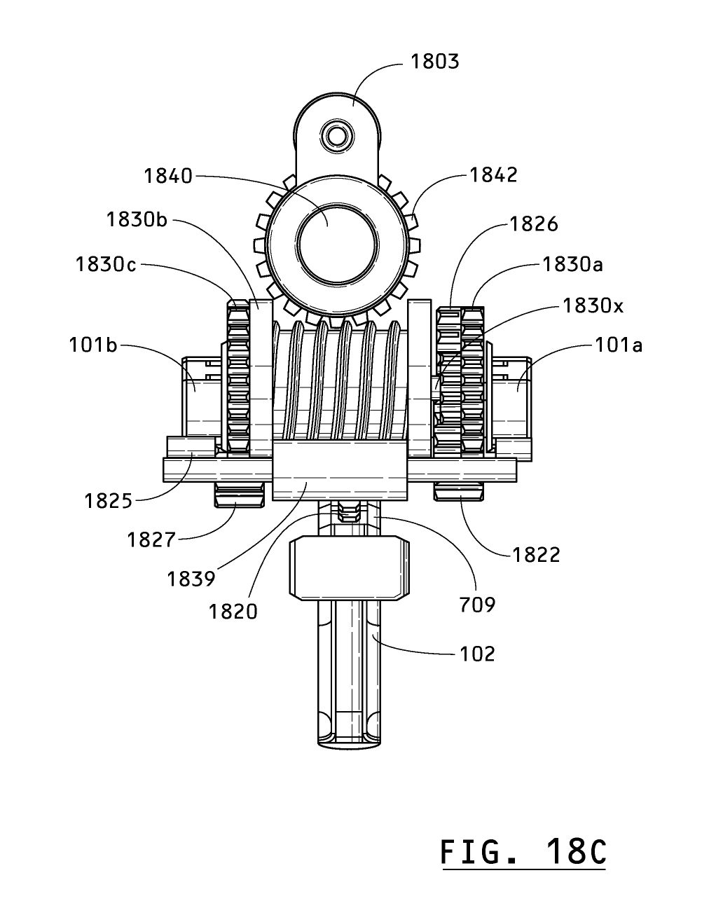

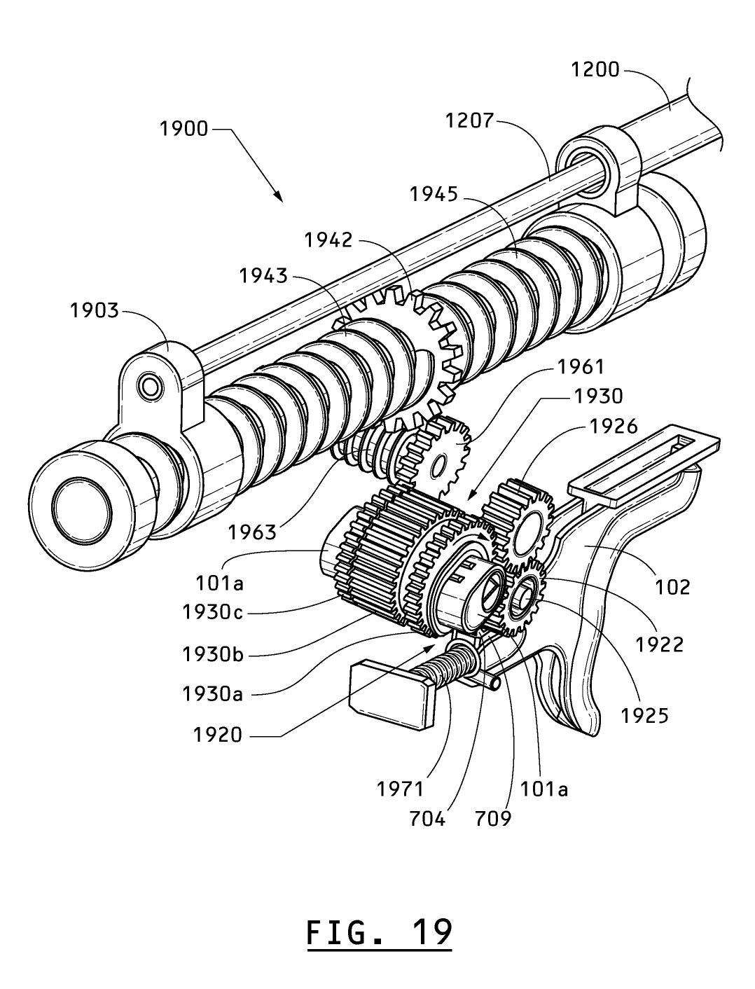

FIGS. 18A-18C and 19 show, respectively, two other embodiments for an internal mechanism 1800 of a stent deployment/resheathing device 100 (where the external appearance of the device may be similar to the embodiment shown in FIG. 1). The lower gears of the embodiments in FIGS. 18A-18C and 19 bear some similarity to those shown and described in FIGS. 3-4. Efficiency of deployment/resheathing motion may be gained by replacing the belt drive with a worm-driven longitudinal threaded shaft drive in these embodiments. FIG. 18A shows a right-side perspective view, FIG. 18B shows a left-side perspective view, and FIG. 18C shows a proximal end plan view of the internal mechanism 1800 (e.g., with an outer cover such as, for example, housing 100a removed to expose the internal components configured to actuate the inner sheath 1802 and outer sheath 1804). Structure and actuation of the mechanism 1800 may be understood with reference to FIGS. 18A-18C, as well as—for the structure and operation of lower gearing portions—to FIGS. 2-4, 7-10, and 11-11A.

A trigger 102 is biased distally (toward the upper-right, in FIG. 18A) by a biasing spring 1871 or other biasing means. The trigger 102 includes a rack 709 with teeth 704 engaging in mechanical communication with the teeth of a central main gear 1820. The central main gear 1820 is disposed rotatably about a pivot axis defined by central main gear shaft 1825 that is oriented perpendicular to the longitudinal proximal-distal axis of the trigger 102. The central main gear shaft 1825 engages a first deployment gear 1822 on its first lateral side and a first resheathing gear 1827 (shown in FIG. 18B; analogous in structure to second drive gear 401 of FIG. 3) on its second/opposite lateral side. The deployment gear 1822 and resheathing gear 1827 share a common pivot/rotation axis with the central main gear 1820. The first deployment gear 1822 includes a clutch mechanism engaged with the central main gear shaft 1825. The clutch mechanism is not shown, but may be embodied in a manner known in the art, such as—for example—as described above with reference to gear 502 and FIG. 2. This clutch mechanism is configured such that the first deployment gear 1822 and resheathing gear 1827 will each rotate only in a single direction while allowing reciprocating rotary motion of the central main gear 1820 in both directions relative thereto with successive generally longitudinal actuations of the trigger 102.

The first deployment gear 1822 is engaged in mechanical communication with a second deployment gear 1826, which is disposed rotatably about a pivot axis that is parallel to axis 1825. The second deployment gear 1826 is engaged in mechanical communication with the deployment gear end 1830a of transition gear assembly 1830. The transition gear assembly 1830 includes a first (deployment) toothed gear end 1830a that is parallel with and affixed by an axle 1830x to a second (resheathing) toothed gear end 1830c. The first (deployment) toothed gear end 1830a and the second (resheathing) toothed gear end 1830c, as well as with the main central gear 1820 and first and second deployment gears 1822, 1826, all rotate about parallel axes that are perpendicular to a plane defined by the body of the trigger 102. A threaded central worm gear portion 1830b is disposed between the first and second toothed gear ends 1830a, 1830c.

The first and second toothed gear ends 1830a, 1830c, separated by the axle 1830x are configured such that only one of them at a time will engage with the threaded central worm gear portion 1830b. In the configuration shown in FIGS. 18A-18C, the second toothed gear end 1830c is engaged with—and the first toothed gear end 1830 is disengaged from—the clutch mechanism of threaded central worm gear portion 1830b (which is configured to rotate gear portion 1830b in a first direction when engaged with gear end 1830a and in an opposite second direction when engaged with gear end 1830c. This configuration provides the reversibility in direction of actuation that may be accomplished by reciprocal longitudinal movement of the trigger 102. A spacer pin 1839 prevents the threaded central worm gear portion 1830b from moving laterally along the rotational axis it shares with the first and second toothed gear ends 1830a, 1830c.

Stated differently, the first and second toothed gear ends 1830a, 1830c are connected by a central axle 1830x extending through the central portion of threaded central worm gear portion 1830b in a manner that allows the first and second toothed gear ends 1830a, 1830c to move laterally (relative to the overall mechanism 1800, which is longitudinally relative to the threaded central worm gear portion 1830b) with actuation of the buttons 101a, 101b in a manner that does not laterally move the threaded central worm gear portion 1830b. The central axle 1830x is disposed along the common pivot/rotation axis of the first and second toothed gear ends 1830a, 1830c and the threaded central worm gear portion 1830b. Engagement of a clutch-face of the gear end 1830a with the threaded central worm gear portion 1830b will effect its rotation in a first direction, while engagement of a clutch-face of the gear end 1830a with the threaded central worm gear portion 1830b will effect its rotation in an opposite second direction.

The central worm gear portion 1830b is configured in engaged mechanical communication with a toothed central driveshaft gear 1842 that is disposed centrally on the driveshaft 1840. The driveshaft 1840 extends along the longitudinal axis of the mechanism 1800, and is configured to rotate about that longitudinal axis. The driveshaft 1840 includes a proximal drive shaft portion 1843 and a distal drive shaft portion 1845. The proximal and distal drive shaft portions 1843, 1845 are threaded. As shown in FIG. 18A, the threading of the proximal drive shaft portion 1843 is opposite the threading of the distal drive shaft portion 1845 (e.g., one is “right-hand-threaded,” while the other is “left-hand-threaded”).

As will be understood with reference to the perspective view shown in FIG. 18A (in view of the rest of the specification), in the position shown, the directional switch 101 may be operated so that distal-to-proximal actuation/depression of the trigger 102 against its biasing spring 1871 will rotate the central main gear 1820 and the first deployment gear 1822 clockwise. The engagement of the first deployment gear 1822 with the second deployment gear 1826 rotates it counterclockwise, which—in turn—rotates clockwise the transition gear 1830. The transition gear's rotation will be translated to clockwise rotation of the driveshaft 1840.

The outer shaft 1200 is longitudinally-movably engaged to the driveshaft 1840 via a distal slide bushing 1805. The inner shaft 1200 is longitudinally-movably engaged to the driveshaft 1840 via a proximal slide bushing 1803. The proximal and distal slide bushings 1803, 1805 are engaged in mechanical communication, respectively, with the proximal and distal drive shaft threaded portions 1843, 1845. This engagement is configured such that clockwise rotation (as viewed along the longitudinal axis from the proximal end as in FIG. 18A) will move the proximal bushing 1803 and inner shaft 1207 distally, and the distal bushing 1805 and outer shaft 1200 proximally in a deployment action by operation of the opposite threading of the proximal and distal shaft portions 1843, 1845. The deployment action may be understood with reference to the transition shown (and discussed herein) from FIG. 15 to FIG. 16, where a stent 301 is extended distally from the shaft. The difference with present embodiment is that the inner shaft 1207 is simultaneously extended distally—pushing the stent 301 distally—while the outer shaft 1200 is withdrawn proximally from its constraining engagement around the stent 301. A stent, such as—for example—a TTS stent, may be releasably attached to at least one of the inner shaft 1207, the outer shaft 1200, or both. Counterclockwise rotation will move the proximal bushing 1803 and inner shaft 1207 proximally and the distal bushing 1805 and outer shaft 1200 distally in a resheathing action, as described below with reference to FIG. 18B.

As described above with reference to other embodiments, the toothed ends 1830a, 1830c of transition gear 1830 may be moved along the longitudinal axis of the transition gear 1830 (without moving central threaded gear portion 1830b) by actuating one or the other of the directional switches 101a, 101b. In the position shown in FIG. 18A, the deployment gears are engaged so as to rotate the first/deployment end of the transition gear 1830a. It will be appreciated that operating the directional switch 101b will move the transition gear 1830 such that its opposite end—second/resheathing transition gear end 1830c will engage the resheathing gear 1827 and operate accordingly.

The mechanism 1800 of delivery device configured for delivering an intraluminal device (such as—for example—a stent) may be characterized as having a lower gear set including the central main gear 1820, at least one deployment gear 1822, and at least resheathing gear 1827 (all three of which share a common pivot/rotation axis of the axle 1825), as well as the second deployment gear 1826. So characterized, the mechanism also includes an upper gear set including the transition gear assembly 1830, which is selectably engageable with the deployment gears 1822, 1826 or the resheathing gear 1827. The upper gear set perpendicularly translates the rotation of the selected lower gear(s) via its worm gear portion 1830b, 1842 into rotation of the driveshaft 1840 about the longitudinal axis of the driveshaft 1840.

Those of skill in the art will appreciate the efficiencies in movement gained by simultaneously advancing and retracting the inner and outer catheters 1207, 1200 for both deployment and resheathing (e.g., with reference to the methods described above and FIGS. 12-16).

FIG. 19 shows a right-side perspective view of the internal mechanism 1900 (e.g., with an outer cover such as, for example, housing 100a of FIG. 1 removed to expose the internal components configured to actuate the inner sheath 1207 and outer sheath 1200). Structure and actuation of the mechanism 1900 may be understood with reference to FIG. 19, as well as—for the lower gearing portions—to FIGS. 2-4, 8-9, 11-12, and 18A-18C.

A trigger 102 is biased distally (toward the upper-right, in FIG. 19) by a biasing spring 1971 or other biasing means. The trigger 102 includes a rack 709 with teeth 704 engaging in mechanical communication with the teeth of a central main gear 1920. The central main gear 1920 is disposed rotatably about a pivot axis 1925 that is oriented perpendicular to the longitudinal proximal-distal axis of the trigger 102. The central main gear 1920 includes a first deployment gear 1922 on its first lateral side and a first resheathing gear on its second/opposite lateral side (not shown, but—like all components of the gear elements numbered in the range 1920-1929—analogous in all respects to the similarly numbered gear elements in FIGS. 18A-18C). The first deployment gear 1922 and resheathing gear share the same pivot axis 1925 as the central main gear 1920. The first deployment gear 1922 includes a clutch mechanism (not shown, but embodied in a manner known in the art, such as—for example—as described above with reference to gear 502 and FIG. 2). This clutch mechanism is configured such that the first deployment gear 1922 (and its unshown resheathing counterpart) will only rotate in a single direction while allowing reciprocating rotary motion of the central main gear 1920 relative thereto with successive generally longitudinal actuations of the trigger 102.

The first deployment gear 1922 is engaged in mechanical communication with a second deployment gear 1926, which is disposed rotatably about a pivot axis that is parallel to axis 1925. The second deployment gear 1926 is engaged in mechanical communication with the deployment gear end 1930a of tripartite transition gear 1930. Transition gear 1930 includes a first (deployment) toothed gear end 1930a that is parallel with a second (resheathing) toothed gear end 1930c, as well as with the main central gear 1920 and first and second deployment gears 1922, 1926, all of which rotate about parallel axes that are perpendicular to a plane defined by the body of the trigger 102. The first and second toothed end gears 1930a, 1930c are separated by a toothed central gear portion 1930b that has a larger diameter than either of the toothed gear ends.

The central toothed gear portion 1930b is configured as engaged mechanical communication with the toothed portion 1961 of a transition worm gear 1960. A threaded gear portion 1963 of the transition worm gear 1960 is engaged with a toothed central driveshaft gear 1942 that is disposed centrally on the driveshaft 1940. The driveshaft 1940 extends along the longitudinal axis of the mechanism 1900, and is configured to rotate about that longitudinal axis. The driveshaft 1940 includes a proximal inner-shaft drive portion 1943 and a distal outer-shaft drive portion 1945. The proximal and distal drive shaft portions 1943, 1945 are threaded.

As will be understood with reference to the perspectives shown in FIG. 19 (in view of the rest of the specification), in the position shown, the directional switch 101 has been operated so that distal-to-proximal actuation/depression of the trigger 102 against its biasing spring 1971 will rotate the central main gear 1920 and the first deployment gear 1922 clockwise. The engagement of the first deployment gear 1922 with the second deployment gear 1926 rotates it counterclockwise, which—in turn—rotates clockwise the tripartite gear 1930. The transition gear's rotation will be translated to clockwise rotation of the driveshaft 1940 via rotation of the transition worm gear 1960.

The outer shaft 1200 is longitudinally-movably engaged to the driveshaft 1940 via a distal slide bushing 1905. The inner shaft 1200 is longitudinally-movably engaged to the driveshaft 1940 via a proximal slide bushing 1903. The proximal and distal slide bushings 1903, 1905 are engaged in mechanical communication, respectively, with the proximal and distal drive shaft threaded portions 1943, 1945. This engagement is configured such that clockwise rotation (as viewed along the longitudinal axis from the proximal end, as in FIG. 18B) will move the proximal bushing 1903 and inner shaft 1207 distally, and the distal bushing 1905 and outer shaft 1200 proximally in a deployment action. Counterclockwise rotation will move the proximal bushing 1903 and inner shaft 1207 proximally and the distal bushing 1905 and outer shaft 1200 distally in a resheathing action.

As described above with reference to other embodiments, the transition gear 1930 may be moved along its longitudinal axis by actuating one or the other of the directional switches 101. In the position shown in FIG. 19, the deployment gears are engaged so as to rotate the first/deployment end of the transition gear 1930a. It will be appreciated that operating the directional switch 101b will move the transition gear 1930 such that its opposite end—second/resheathing end 1930c will engage the resheathing gear and operate accordingly.

Those of skill in the art will appreciate the efficiencies in movement gained by simultaneously advancing and retracting the inner and outer catheters 1207, 1200 for both deployment and resheathing (e.g., with reference to the methods and structures described above and FIGS. 12-16). For example, a positional state of a stent 301 to be deployed may correspond to that shown in FIG. 15, while the handle/trigger position corresponds to that shown in FIG. 13, and—after an actuation of the trigger to the position shown in FIG. 14, the stent may be further deployed to the state shown in FIG. 16 when the deployment gear set is engaged. Of course, the reverse may be true if the resheathing gear set is engaged.

The above described deployment and resheathing methods may also be utilized for TTS stents such as colonic or duodenal stents. Deployment or resheathing of such TTS stents preferably would involve using a reinforced outer sheath in place of a non-reinforced outer sheath and a retaining loop assembly and lockwire in place of the bilumen tubing/suture wire described in FIGS. 13-16 (see, e.g., the structural components disclosed in U.S. Pat. App. Publ. 2010/0168834).

The above drawing figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. Furthermore, the advantages described above are not necessarily the only advantages of the invention, and it is not necessarily expected that all of the described advantages will be achieved with every embodiment of the invention.

We claim:

1. A delivery device for delivering an intraluminal device, comprising:
a gear mechanism comprising a first gear set and a second gear set;
the gear mechanism configured to selectably mechanically couple a threaded drive shaft to one of the first gear set and the second gear set via a threaded worm gear; and
an outer elongate shaft disposed slidably over an inner elongate shaft, the outer elongate shaft comprising a mechanical communication connection with a first portion of the threaded drive shaft; and
the inner elongate shaft comprising a mechanical communication connection with a second portion of the threaded drive shaft;
wherein the gear mechanism is configured such that actuation of the first gear set rotates the threaded drive shaft in a first direction, and actuation of the second gear set rotates the threaded drive shaft in a second direction that is opposite the first direction;
wherein the first portion of the threaded drive shaft is threaded opposite the second portion of the threaded drive shaft; and
wherein the mechanical communication connection of the outer shaft with the first portion of the threaded drive shaft and the mechanical communication connection of the inner shaft with the portion of the threaded drive shaft are configured to move the inner and outer shafts in opposite longitudinal directions from each other upon rotation of the threaded drive shaft.

2. The delivery device of claim 1, further comprising a trigger member disposed in mechanical communication with the gear mechanism and configured to be reciprocally actuated in a generally longitudinal fashion.

3. The delivery device of claim 2, wherein the first gear set comprises a main central gear contacting the trigger member in a manner configured to rotate the main central gear upon actuation of the trigger member.

4. The delivery device of claim 3, wherein the first gear set further comprises a pair of gears configured to transmit a rotary motion of the main central gear to the worm gear.

5. The delivery device of claim 2, configured such that motion of the trigger parallel to a longitudinal axis of the threaded drive shaft is translated by the worm gear to rotary motion of the threaded drive shaft about said longitudinal axis.

6. The delivery device of claim 2, further comprising a clutch mechanism of the first gear set configured to provide unidirectional rotation upon reciprocal actuation of the trigger member.

7. The delivery device of claim 1, further comprising a stent.

8. The delivery device of claim 7, wherein the stent is releasably attached to the outer shaft.

9. The delivery device of claim 7, wherein the stent is releasably attached to the inner shaft.

10. The delivery device of claim 7, wherein the stent is disposed around the inner shaft and is at least partially circumferentially constrained by the outer shaft.

11. The delivery device of claim 1, wherein the mechanical communication connection of the outer shaft with a first portion of the threaded drive shaft comprises a bushing configured to travel longitudinally along the first portion of the threaded drive shaft.

12. The delivery device of claim 1, wherein the mechanical communication connection of the inner shaft with a second portion of the threaded drive shaft comprises a bushing configured to travel longitudinally along the second portion of the threaded drive shaft.

13. A delivery device configured for delivering an intraluminal device, comprising:
a threaded driveshaft comprising a longitudinal axis;
a gear mechanism comprising a lower gear set comprising at least one deployment gear and at least one resheathing gear sharing a common rotation axis, and an upper gear set comprising a worm gear configured to perpendicularly translate rotation of a selected one of the at least one deployment gear or at least one resheathing gear with which it is engaged into rotation of the driveshaft about its longitudinal axis;
an outer elongate shaft disposed slidably over an inner elongate shaft, the outer elongate shaft comprising a mechanical communication connection with a first portion of the threaded drive shaft; and
the inner elongate shaft comprising a mechanical communication connection with a second portion of the threaded drive shaft;
wherein the gear mechanism is configured such that actuation of the lower gear set rotates the threaded drive shaft in a first direction when the at least one deployment gear is engaged with the worm gear, and actuation of the lower gear set rotates the threaded drive shaft in a second direction that is opposite the first direction when the at least one resheathing gear is engaged with the worm gear;
wherein the first portion of the threaded drive shaft is threaded opposite the second portion of the threaded drive shaft; and
wherein the mechanical communication connection of the outer shaft with the first portion of the threaded drive shaft and the mechanical communication connection of the inner shaft with the portion of the threaded drive shaft are configured to move the inner and outer shafts in opposite longitudinal directions from each other upon rotation of the threaded drive shaft.

14. The delivery device of claim 13, further comprising a trigger member disposed in mechanical communication with the lower gear set and configured to be reciprocally actuated in a generally longitudinal fashion to rotate the at least one deployment gear and the at least one resheathing gear about a common axis.

15. The delivery device of claim 14, wherein the mechanical communication comprises a main central gear sharing the common rotation axis and contacting the trigger member in a manner configured to rotate the main central gear upon actuation of the trigger member.

16. The delivery device of claim 13, further comprising a stent.

17. The delivery device of claim 16, wherein the stent is configured as a through-the-scope stent.

18. The delivery device of claim 16, wherein the stent is releasably attached to the inner shaft.

19. The delivery device of claim 16, wherein the stent is disposed around the inner shaft and is at least partially circumferentially constrained by the outer shaft.

20. The delivery device of claim 1, wherein the mechanical communication connection of the outer shaft with a first portion of the threaded drive shaft comprises a bushing configured to travel longitudinally along the first portion of the threaded drive shaft.
